(19) United States
(12) Patent Application Publication
(10) Pub. No.: US 2024/0157175 A1
(43) Pub. Date: May 16, 2024

(54) MEDICAL TREATMENT FACILITY WITH RING GANTRY AND ROTATABLE PATIENT COUCH

(71) Applicant: Siemens Healthcare GmbH, Erlangen (DE)

(72) Inventor: Wolfgang NEUBER, Eschenbach (DE)

(73) Assignee: Siemens Healthcare GmbH, Erlangen (DE)

(21) Appl. No.: 18/505,840

(22) Filed: Nov. 9, 2023

(30) Foreign Application Priority Data
Nov. 10, 2022 (EP) 22206720.9

(51) Int. Cl. A61N 5/10

(52) U.S. Cl. CPC A61N 5/1081

(57) ABSTRACT

A medical treatment facility has a ring gantry and a patient couch. The patient couch includes a base element and an intermediate element supported via a proximal support element and a distal support element on the base element, the proximal support element and the distal support element being outside a volume surrounded by the ring gantry and on a same side of the ring gantry, the proximal support element being nearer to the ring gantry than the distal support element, the intermediate element being guidable via at least one of the proximal support element or the distal support element such that the intermediate element rotates about a vertical axis, the vertical axis lies at a first side of the proximal support element or on a second side of the proximal support element.

### CROSS-REFERENCE TO RELATED APPLICATION(S)

The present application claims priority under 35 U.S.C. § 119 to European Patent Application No. 22206720.9, filed Nov. 10, 2022, the entire contents of which are incorporated herein by reference.

Regardless of the grammatical gender of a specific term, persons with male, female or other gender identity are included as well.

### SUMMARY

One or more example embodiments of the present invention provides opportunities via which, in a simple, low-cost and reliable manner, a rotation of the intermediate element about a vertical axis can be realized, wherein the vertical axis can be brought as near as possible to the isocenter of the ring gantry, where possible can even be placed in the isocenter.

The object is achieved by a medical treatment facility with the features of claim 1. Advantageous embodiments of the medical treatment facility are the subject matter of the dependent claims 2 to 10.

### BRIEF DESCRIPTION OF THE DRAWINGS

Characteristics, features and advantages of this invention and also the way in which these are achieved will become clearer and easier to understand in conjunction with the description of the exemplary embodiments given below, which will be explained in greater detail in conjunction with the drawings. In the drawings here, as schematic diagrams:

FIG. 1 shows a medical treatment facility in a perspective view,

FIG. 2 shows a base element and a floor-level plate of an intermediate element in a non-rotated state,

FIG. 3 shows the base element and the floor-level plate from FIG. 2 in a rotated state,

FIG. 4 shows a patient couch from the side,

FIG. 5 shows a perspective view of a base element and a floor-level plate,

FIG. 6 shows a detail from FIG. 5,

FIG. 7 shows a detail from FIG. 6 from the side,

FIG. 8 shows an Omega drive,

FIG. 9 shows a ring gantry and a base element and

FIG. 10 shows a ring gantry and a further base element.

### DETAILED DESCRIPTION

For applications in radiotherapy the patient couch must often be rotated about a vertical axis in order to compensate for positioning errors of the target volume that is to be irradiated. The target volume is as a rule a specific region of the body of a patient. In some cases only a small angle of rotation is possible (a maximum of 5° for example). In such cases it is exclusively a correction of positioning errors that can be undertaken by rotating the patient couch. In other cases larger angles of rotation can also be set (of up to 30° for example). In such cases there can also be an irradiation of the target volume from markedly different angles (in relation to the target volume) by the rotation, so that the volume surrounding the target volume is subjected to less stress.

The optimum is a rotation about a vertical axis passing through the isocenter of the ring gantry. This is because no translational relocation of the target volume is then produced by the rotation.

Theoretically it is conceivable to achieve a corresponding positioning of the vertical axis by a part of a support structure running around in circular curved guide elements on both sides of the ring gantry, wherein the two parts of the support structure are able to be rotated about a common axis of rotation—namely the vertical axis—via the two guide elements. In this case the support would have to be arranged on the support structure on which the couch board is able to be moved linearly. This solution, which is possible in principle is however associated with significant constructional disadvantages and will therefore not be adopted as a rule.

In practice guide structures for making possible a rotation are much rather located on just one side of the ring gantry, namely on the side on which the patient couch is located.

An important criterion for the design of the patient couch is inter alia its stability, in order to be able to achieve a reproducible positioning of the patient or of the target volume of the patient. The greater the rigidity of the patient couch is, the more accurately can the positioning be carried out.

Arranging the intermediate element rotatably on the base element via a pivot bearing is known. In this embodiment however two mutually conflicting requirements occur. This is because on the one hand the pivot bearing should have a diameter that is as large as possible, since the stability of the pivot bearing increases with the diameter. On the other hand the pivot bearing should have a diameter that is as small as possible, since the location of the vertical axis in the horizontal plane is defined by the pivot bearing and, due to the diameter of the pivot bearing, is further away from the isocenter, the greater the diameter of the pivot bearing is. The further the vertical axis is away from the isocenter however, the greater is also the translational movement of the target volume occurring during a rotation.

In practice the diameter of the pivot bearing is mostly selected to be relatively large in order to guarantee the necessary stability. The disadvantage of the relatively large translational relocation of the target volume associated therewith is taken into account. A further disadvantage of the solution adopted in practice consists of such a pivot bearing being costly.

One or more example embodiments of the present invention arises from a medical treatment facility, especially a radiotherapy facility,
wherein the medical treatment facility has a ring gantry, which is embodied as an upright ring running around a horizontal longitudinal direction, and has an isocenter,
wherein the medical treatment facility has a patient couch, which is arranged, viewed in the longitudinal direction, in front of the ring gantry,
wherein the patient couch has a base element, an intermediate element, a support and a couch board,
wherein the base element is attached to the ring gantry, so that it is not able to be moved or is exclusively able to be moved in a horizontal transversal direction orthogonal to the longitudinal direction,
wherein the support is arranged on the intermediate element,
wherein the couch board is able to be moved on the support in a horizontal direction of movement at least essentially running in the longitudinal direction.

In accordance with one or more example embodiments of the present invention, a method of operation of the type stated at the outset is embodied such that,
the intermediate element is supported on the base element via a proximal and a distal support element,
both support elements are arranged outside of a volume surrounded by the ring gantry and, viewed from the ring gantry outwards, are arranged on the same side of the ring gantry,
the proximal support element is arranged nearer to the ring gantry than the distal support element,
the intermediate element is guided via at least one of the two support elements in such a way that it rotates about a vertical axis, and
the vertical axis, viewed at the proximal guide element or viewed from the distal guide element outwards, lies on the other side of the proximal guide element, in particular at the isocenter of the ring gantry.

This embodiment on the one hand enables a simple construction to be achieved, on the other hand enables a reliable guidance of the intermediate element to be achieved and, over and above this, enables the vertical axis to be brought at least into the vicinity of the isocenter of the ring gantry.

It is possible for the proximal support element to be embodied as a guide element, which guides the intermediate element on an inner arc of a circle with an inner arc radius about the vertical axis, and for the distal support element to be embodied as a guide element, which guides the intermediate element on an outer arc of a circle with an outer circle radius about the vertical axis. This embodiment has the advantage that the vertical axis can be brought into the isocenter of the ring gantry.

As an alternative it is possible for the proximal support element to be embodied as a pivot bearing, through the center point of which the vertical axis passes. This embodiment has the advantage that the actual guidance of the intermediate element is brought about solely by the proximal support element. The distal guide element essentially only assumes the task of a support.

As another alternative it is possible for the proximal support element to be embodied as a point-type attachment element, through which the vertical axis passes and for the intermediate element to be attached rotatably to it. In this case the vertical axis lies directly in the region of the proximal support element and thus outside the volume enclosed by the ring gantry. The proximal support element in this case is however very simple and also very small, so that the vertical axis can be guided to the edge of the ring gantry.

Various embodiments are possible for the internal layout of the intermediate element. For example the intermediate element can have a telescopic lifting column, via which the support can be raised and lowered in the vertical direction. Furthermore an embodiment as a kind of articulated knee joint is possible. Other embodiments are also possible. In a preferred embodiment of the treatment facility there is provision however,
for the intermediate element to have a floor-level plate facing towards the base element,
for the support of the couch board to be connected via a scissor mechanism of the intermediate element to the floor-level plate,
for the scissor mechanism to have two scissor arms, which are linked to each other by an articulated joint in the middle, so that the two scissor arms form an X,
for a lower end point of the one scissor arm to be fixed to the floor-level plate and for a lower end point of the other scissor arm to be guided in a guide of the floor-level plate, so that a distance between the lower end point of the other scissor arm and the ring gantry and thereby a vertical distance between the support of the couch board and the base element is adjustable,
for the lower end point of the scissor arm fixed to the floor-level plate to be arranged nearer to the ring gantry than to the lower end point of the scissor arm guided in the guide of the floor-level plate.

This embodiment makes a height adjustment of the support of the couch board possible in a simple and reliable way. Furthermore a favorable guidance of force is produced by this embodiment.

Preferably the lower end point of the scissor arm fixed to the floor-level plate is arranged in the region of the proximal support element. This in particular enables the weight to be transmitted in a simple way from the lower end point of the scissor arm fixed to the floor-level plate to the floor-level plate and from there to the base element.

Furthermore the lower end point of the scissor arm guided in the guide of the floor-level plate is arranged in the region of the distal support element in the state, in which the distance between the lower end point of the scissor arm guided in the guide of the floor-level plate and the ring gantry is minimal. In normal operation, when the support of the couch board is moved upwards and the highest loads occur, this in particular enables the tensile force occurring to be transmitted in a simple manner from the lower end point of the scissor arm guided in the guide of the floor-level plate to the floor-level plate and from there to the base element.

For acceptance of forces directed in the vertical direction, which act from the intermediate element on the distal support element, the distal support element preferably has a fixing plate extending in the horizontal plane, which interacts with the counter elements arranged on the intermediate element. This makes possible in a simple manner a transmission of the forces acting in this area from the intermediate element to the distal support element.

Preferably the counter elements comprise upper counter elements and lower counter elements, wherein the upper counter elements transmit compressive forces acting from above to the fixing plate and the lower counter elements transmit tensile forces acting from below to the fixing plate. The transmission of the forces is thereby designed especially simply. The counter elements can in particular be embodied as rollers that roll over the fixing plate.

Preferably the upper counter elements and/or the lower counter elements are arranged on the intermediate element adjustable in the vertical direction.

If only the upper or only the lower counter elements are adjustable, play can be rectified by the corresponding adjustment, so that both the upper and also the lower counter elements rest smoothly on the fixing plate (and where necessary roll over it). Preferably however both the upper and also the lower counter elements are adjusted and are adjusted independently of one another. This enables, not only—as previously—play to be rectified, but above and beyond this also enables an adjustment of the intermediate element to be undertaken, in particular in such a way that the direction of movement is exactly horizontal.

Due to the situation that in particular a tilting moment about the lower end point of the scissor arm fixed to the floor-level plate is captured via the counter elements, the proximal support element is dimensioned (sc. small enough) so that, if the distal support element were not present, it would be destroyed by the forces occurring during operation, in particular shear forces or bending moments. This enables the proximal support element to be manufactured at very low cost.

Preferably a drive for rotation of the intermediate element about the vertical axis is embodied as an Omega drive. This in particular enables the drive for rotation of the intermediate element about the vertical axis to be arranged in the base element. This simplifies access to and also supply of energy to this drive.

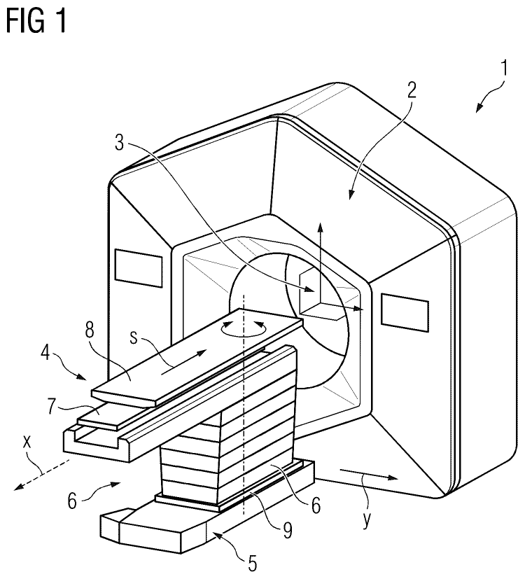

In accordance with FIG. 1 a medical treatment facility 1 has a ring gantry 2. The ring gantry 2 can be seen as being embodied as an upright ring running around a horizontal longitudinal direction x. The ring gantry 2 has an isocenter 3. The ring gantry 2 and thus the medical treatment facility 1 as a whole can be embodied for a treatment (therapy) of a patient by irradiation, i.e. as a radiotherapy facility.

The medical treatment facility 1, in addition to the ring gantry 2, has a patient couch 4. The patient couch 4 is arranged, viewed in the longitudinal direction x, in front of the ring gantry 2. The patient couch 4 has a base element 5, an intermediate element 6, a support 7 and a couch board 8.

The base element 5 is attached, in accordance with FIG. 1, to the ring gantry 2. It is possible for the attachment of the base element 5 to be rigid, i.e. immovable and fixed. According to the diagram in FIG. 1 however, it is preferred as a rule for the base element 5 to be attached to the ring gantry 2 in such a way that the base element 5 is able to be displaced in a transverse direction y. The transverse direction y likewise runs horizontally, but orthogonal to the longitudinal direction x. There is generally no provision for a further displaceability of the base element 2.

The support 7 is arranged on the intermediate element 6. The couch board 8 can be moved on the support 7 in a direction of movement s. The direction of movement s runs horizontally, and does so at least essentially in the longitudinal direction x. A patient can be supported on the couch board 8 (not shown in the figure). The movement of the couch board 8 enables a region of the patient (provided he is supported on the couch board 8) to be arranged in the isocenter 3 of the ring gantry 2. The direction of movement s is variable within the horizontal plane. This will become clearer from explanations given below.

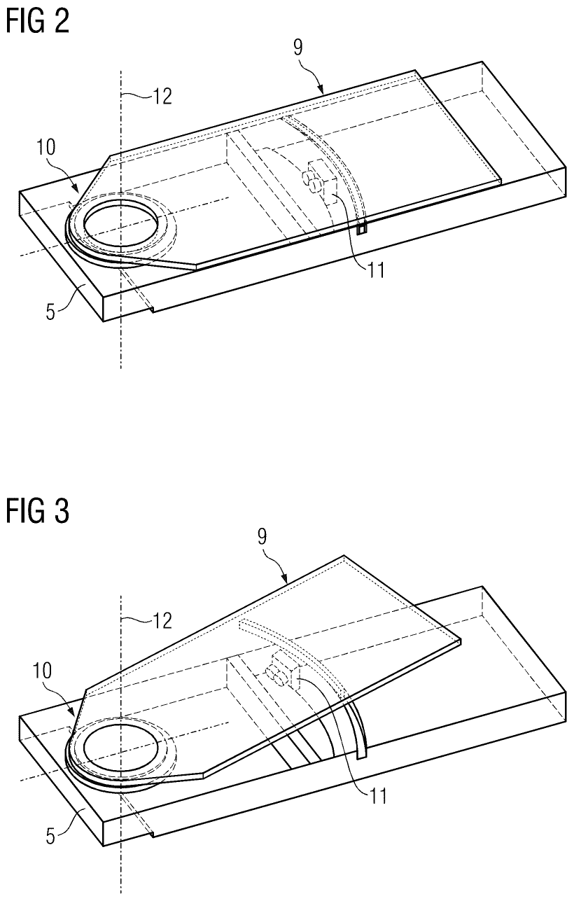

FIGS. 2 and 3 show the base element 5 and a floor-level plate 9 of the intermediate element 6.

In accordance with FIGS. 2 and 3 the floor-level plate 9 of the intermediate element 6 is supported on the base element 5 via two support elements 10, 11. Both support elements 10, 11 are arranged outside a volume enclosed by the ring gantry 2. Furthermore both support elements 10, 11, viewed from the ring gantry 2 outwards, are arranged on the same side of the ring gantry 2. The one support element 10 is arranged nearer to the ring gantry 2 than the other support element 11. The support element 10 arranged nearer to the ring gantry 2 is referred to below as the proximal element 10, the support element 11 further from the ring gantry 2 as the distal support element 11.

The intermediate element 6 is guided via at least one of the two support elements 10, 11 in such a way that it rotates about a vertical axis 12. This will become clearer from explanations given below. FIG. 2 shows the base element 5 and the floor-level plate 9 in the non-rotated state of the floor-level plate 9, FIG. 3 shows them in the rotated state of the floor-level plate 9.

The vertical axis 12 in the embodiment of FIGS. 2 and 3 lies at the proximal guide element 10. For example, in the embodiment in accordance with FIG. 2 the proximal support element 10 is embodied as a pivot bearing. In this case the vertical axis 12 passes through the center point of the pivot bearing. In other embodiments the vertical axis 12, viewed from the distal guide element 11, can also lie on the far side of the proximal guide element 10, so that the proximal guide element 10 thus lies between the distal guide element 11 and the vertical axis 12. In a suitable embodiment the vertical axis 12 can even lie in the isocenter 3 of the ring gantry 2.

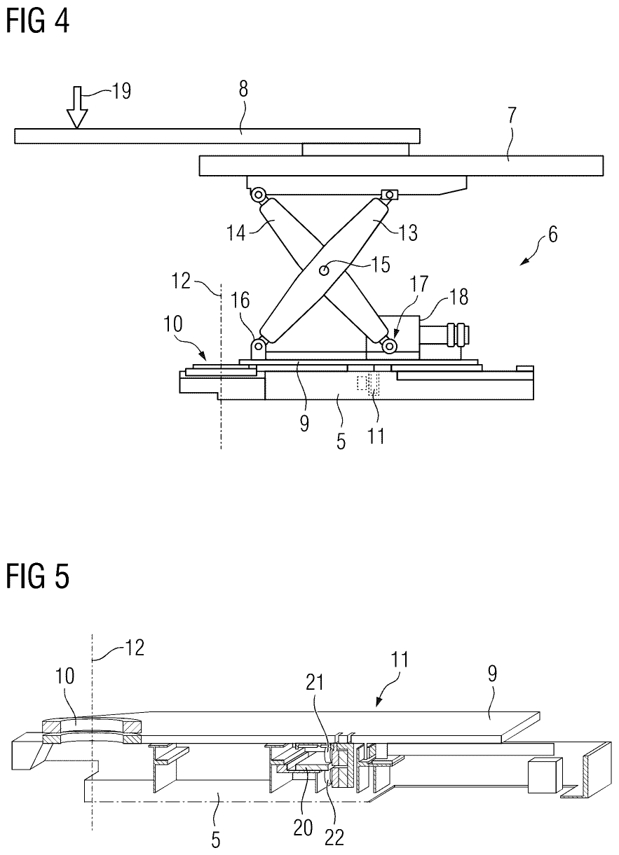

The intermediate element 6 has a mechanism via which the support 7 of the couch board 8 can be adjusted in the vertical direction. In accordance with the exemplary embodiment (see FIG. 4) the intermediate element 6, in addition to the floor-level plate 9, has a scissor mechanism with two scissor arms 13, 14 for this purpose, wherein the scissor arms 13, 14 are connected to each other by an articulated joint in the middle. The hinge point is labeled in FIG. 4 with the reference number 15. Through the hinged connection the two scissor arms 13, 14 form an X. The floor-level plate 9 is arranged towards the base element 5. The scissor mechanism is arranged above the floor-level plate 9.

The support 7 of the couch board 8 is connected to the floor-level plate 9 via the scissor mechanism. In concrete terms a lower end point 16 of the one scissor arm 13 is fixed to the floor-level plate 9. A lower end point 17 of the other scissor arm 14 (hidden in FIG. 4) is guided in a guide of the floor-level plate 9 (likewise hidden in FIG. 4). The element hiding it is a drive 18 for the corresponding adjustment of this lower end point 17. Through corresponding adjustment of the location of this end point 17 a distance between this lower end point 17 and the ring gantry 2 can thus be set. According to the setting of this distance a vertical distance between the support 7 of the couch board 8 and the base element 5 is thus also adjustable at the same time. According to the diagram in FIG. 4 the lower end point 16 of the scissor arm 13 fixed to the floor-level plate 9 is arranged nearer to the ring gantry 2 than the lower end point 17 of the scissor arm 14 guided in the guide of the floor-level plate 9.

Preferably, as can also be seen from FIG. 4, the lower end point 16 of the scissor arm 13 fixed to the floor-level plate 9 is arranged in the area of the proximal support element 10. Furthermore, as can likewise be seen from FIG. 4, the lower end point 17 of the other scissor arm 14 is preferably arranged in the region of the distal support element 11 in the state, in which the distance between this lower end point 17 and the ring gantry 2 is minimal (thus as a result the support 7 and thus also the couch board 8 are raised as far as possible).

When the patient is positioned in the isocenter 3 of the ring gantry 2, the center of gravity of the couch board 8 is mostly arranged outside the area in which the support 7 is connected to the scissor mechanism. The weight force acting at the center of gravity is indicated in FIG. 4 by an arrow 19. The tip of the arrow 19 points to the center of gravity. Due to the lever effect, significant compressive loads can thus act on the distal support element 11. If conversely the couch board 8, and thus also the patient, are not moved into the isocenter 3 (for example because the patient wishes to lay down on the couch board 8 or get up from it at this point), the couch board 8 is located—in relation to the diagram in FIG. 4—significantly further to the right. In this case significant compressive loads act on the distal support element 11. In both cases the forces thus act in the vertical direction.

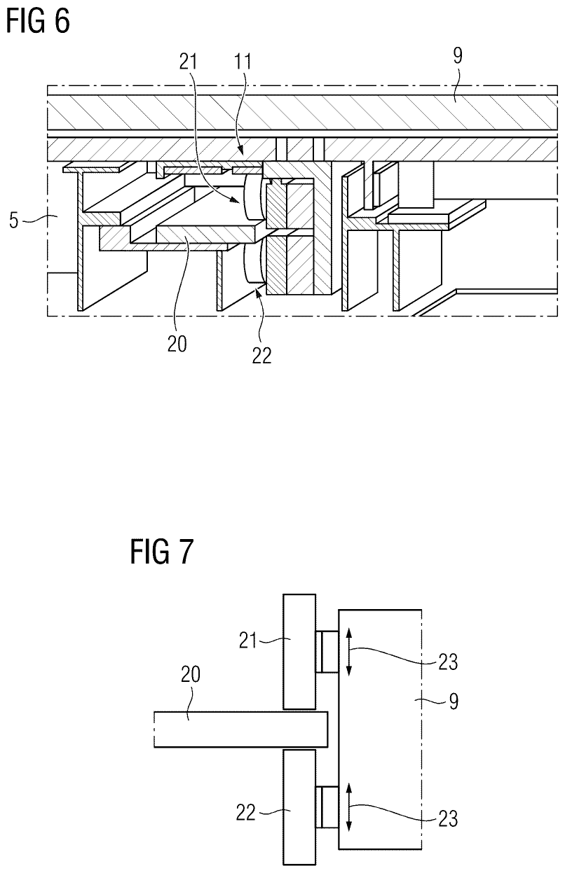

For accommodating such forces directed in a vertical direction, which act from the intermediate element 6 on the distal support element 11, the distal support element 11, according to the diagram in FIGS. 5 and 6, has a fixing plate 20. FIG. 6 shows the area marked in FIG. 5 enclosed by a dotted and dashed line. The fixing plate 20 extends in the horizontal plane. The fixing plate 20 acts with counter elements 21, 22, which are arranged on the intermediate element 6 (more precisely: on the floor-level plate 9). The counter elements 21, 22 can in particular be embodied as rollers that roll over the fixing plate 20.

The counter elements 21 are arranged above the fixing plate 20 and are therefore upper counter elements. The counter elements 21 are thus capable of transmitting compressive forces acting from above to the fixing plate 20. Conversely the counter elements 22 are arranged below the fixing plate 20 and are therefore lower counter elements. The counter elements 22 are thus capable of transmitting tensile forces acting from below to the fixing plate 20.

According to the diagram in FIG. 7 the upper counter elements 21 and/or the lower counter elements 22 are arranged on the intermediate element 6 (more precisely: on the floor-level plate 9), adjustable in a vertical direction. The adjustability is indicated in FIG. 7 by corresponding arrows 23.

Because of the situation that two support elements 10, 11 are present at an appreciable distance from each other, the proximal support element 10 can in particular be dimensioned in such a way that it would be destroyed by the forces occurring during operation—in particular shear forces or bending moments—if the distal support element 11 were not present.

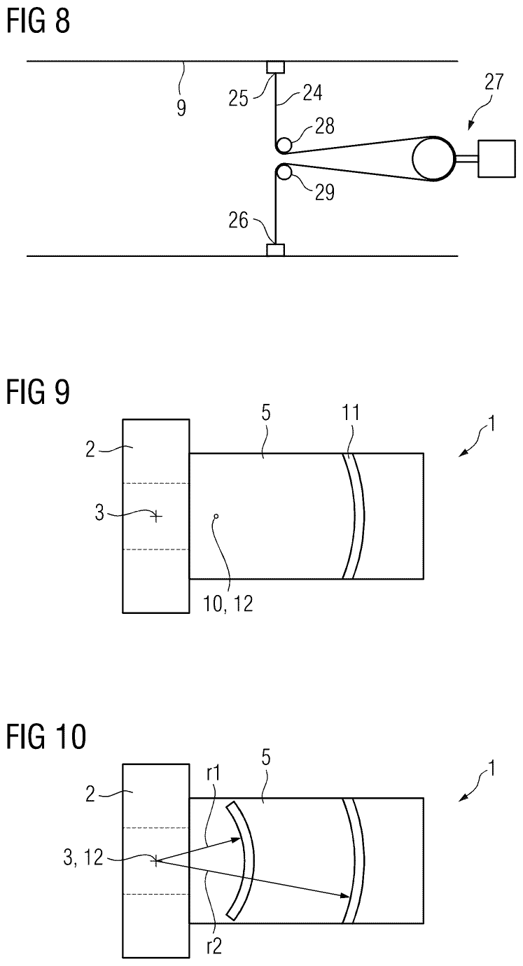

The floor-level plate 9 (and all elements of the patient couch 4 arranged above the floor-level plate) about the vertical axis 12 are rotated in accordance with FIG. 8 via what is known as an Omega drive. With an Omega drive a flexible force transmission means 24 is present, for example a v-belt, a toothed belt or a chain. Ends 25, 26 of the force transmission means 24 are fixed to the element to be moved, here to the floor-level plate 9. The force transmission means 24 is fed to an actual drive 27 via pulleys 28, 29. Both the drive 27 and also the pulleys 28, 29 are fixed to that element, in relation to which the element to be moved is to be moved. In the present example the drive 27 and the pulleys 28, 29 are arranged on the base element 5. If the drive 27 rotates forwards, then it reduces the length between the force transmission means 24 and the one end 25 by a specific amount and increases the length between the force transmission means 24 and the other end 26 by the same amount. If the drive rotates 27 backwards, the opposite is the case. Since the length of the force transmission means 24 from drive 27 to the pulleys 28, 29 is constant, the ends 25, 26 and with them the floor-level plate 9 must move by this amount.

An embodiment of the present invention has been explained above in conjunction with FIGS. 2 to 8, in which the proximal support element 10 is embodied as a pivot bearing and the distal support element 11 must essentially only accommodate vertical forces. Other embodiments are also possible, however.

Thus for example, according to the diagram in FIG. 9, the proximal support element 10 can be embodied as a point-type attachment element, to which the intermediate element 6 is attached rotatably. For example, the proximal support element 10 can be embodied as a receptacle for a bolt of the floor-level plate 9. The inverse embodiment is also possible. In any case, with an embodiment of the proximal support element 10 as a point-type attachment element, the vertical axis 12 passes through the proximal support element 10.

It is furthermore possible that the proximal support element 10 is embodied according to the diagram in FIG. 10 as a guide element, which guides the intermediate element 6 (or the floor-level plate 9) on an inner circle arc with an inner arc radius r1 about the vertical axis 12. In this case the distal support element 11 is embodied according to the diagram in FIG. 10 as a guide element, which guides the intermediate element 6 (or the floor-level plate 9) on an outer circle arc with an outer arc radius r2 about the vertical axis. The two arc radii r1, r2 are matched to one another in such a way that they are both pivoted about the same point, i.e. about the (common) vertical axis 12. In particular in the case of the embodiment in accordance with FIG. 10 the vertical axis 12, viewed from the distal guide element 11, can lie on the other side of the proximal guide element 10, if necessary, even in the isocenter 3.

In summary, one or more example embodiments of the present invention relates to the following subject matter:

A medical treatment facility 1 has a ring gantry 2, which is embodied as an upright ring running around a horizontal longitudinal direction x and having an isocenter 3. The medical treatment facility 1 furthermore has a patient couch 4 that, viewed in the longitudinal direction x, is arranged in front of the ring gantry 2 and has a base element 5, an intermediate element 6, a support 7 and a couch board 8. The base element 5 is attached to the ring gantry 2, so that it is not able to be moved or is exclusively able to be moved in a horizontal transverse direction y orthogonal to the longitudinal direction x. The support 7 is arranged on the intermediate element 6, the couch board 8 on the support 7 in a horizontal direction of movement s at least running in the longitudinal direction x. The intermediate element 6 is supported via a proximal and a distal support element 10, 11 on the base element 5, wherein the proximal support element 10 is arranged nearer to the ring gantry 2 than the distal support element 11. Both support elements 10, 11 are arranged outside a volume surrounded by the ring gantry 2 and, viewed from the ring gantry 2, on the same side of the ring gantry 2. The intermediate element 6 is guided via at least one of the two support elements 10, 11 in such a way that it rotates about a vertical axis 12. The vertical axis 12 lies at the proximal guide element 10 or, viewed from the distal guide element 11, on the other side of it.

One or more example embodiments of the present invention has many advantages. First of all a mechanically simple and robust, low-cost, realization of the rotatability of the couch board 8 about the vertical axis 12 is produced. Such a realization is possible with off-the-shelf components. If the vertical axis 12 passes through the isocenter 3 of the ring gantry 2, the—actually desired—rotation about the isocenter 3 can be realized directly. In the other cases the vertical axis 12 can be placed near to the isocenter 3 of the ring gantry 3. The translational offset that is produced during rotation of the intermediate element 6 about the vertical axis 12 is thereby small. Where these types of offsets are to be compensated for by translational movements, the extent to which these types of translational movements are necessary is correspondingly small. As a result of the distance of the two support elements 10, 11 from one another a high stability of the patient couch 4 and its components can be achieved.

Although the present invention has been disclosed in the form of preferred embodiments and variations thereon, it will be understood that numerous additional modifications and variations could be made thereto without departing from the scope of the invention. For the sake of clarity, it is to be understood that the use of “a” or “an” throughout this application does not exclude a plurality, and “comprising” does not exclude other steps or elements. The expression “a number of” means “at least one”. The mention of a “unit” or a “device” does not preclude the use of more than one unit or device. The expression “a number of” has to be understood as “at least one”.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the invention has been illustrated and described in greater detail by the preferred exemplary embodiment, the invention is not restricted by the disclosed examples and other variations can be derived herefrom by the person skilled in the art, without departing from the scope of protection of the invention.

1. A medical treatment facility, comprising:
a ring gantry, the ring gantry being an upright ring extending around a horizontal longitudinal direction, the ring gantry an isocenter; and
a patient couch arranged at a first side of the ring gantry along the longitudinal direction, the patient couch including,
a base element attached to the ring gantry such that the base element is not movable or is exclusively able to be moved in a horizontal transverse direction orthogonal to the longitudinal direction,
an intermediate element supported via a proximal support element and a distal support element on the base element, the proximal support element and the distal support element being outside a volume surrounded by the ring gantry and on a same side of the ring gantry, the proximal support element being nearer to the ring gantry than the distal support element, the intermediate element being guidable via at least one of the proximal support element or the distal support element such that the intermediate element rotates about a vertical axis, the vertical axis lies at a first side of the proximal support element or on a second side of the proximal support element,
a support on the intermediate element, and
a couch board, the couch board being movable on the support in a horizontal direction of movement running at least essentially in the longitudinal direction.

2. The treatment facility of claim 1, wherein
the proximal support element is a guide element, which guides the intermediate element on an inner circle arc with an inner arc radius about the vertical axis, and the distal support element is a guide element, which guides the intermediate element on an outer circle arc with an outer arc radius about the vertical axis, or
the proximal support element is a pivot bearing, through a center point of which the vertical axis passes, or
the proximal support element is a point-type attachment element, through which the vertical axis passes and to which the intermediate element is rotatably attached.

3. The treatment facility of claim 1, wherein
the intermediate element includes a floor-level plate towards the base element,
the support of the couch board is connected height-adjustably to the floor-level plate via a scissor mechanism of the intermediate element,
the scissor mechanism includes two scissor arms which are connected to one another by an articulated joint in a middle of the scissor mechanism such that the two scissor arms form an X,
a lower end point of one scissor arm is fixed to the floor-level plate and a lower end point of the other scissor arm is guided in a guide of the floor-level plate such that a distance between the lower end point of the other scissor arm and the ring gantry and a vertical distance between the support of the couch board and the base element is settable, and
the lower end point of the scissor arm fixed to the floor-level plate is nearer to the ring gantry than the lower end point of the scissor arm guided in the guide of the floor-level plate.

4. The treatment facility of claim 3, wherein the lower end point of the scissor arm fixed to the floor-level plate is in an area of the proximal support element.

5. The treatment facility of claim 3, wherein the lower end point of the scissor arm guided in the guide of the floor-level plate is in an area of the distal support element in a state in which the distance between the lower end point of the scissor arm guided in the guide of the floor-level plate and the ring gantry is reduced.

6. The treatment facility of claim 1, wherein the distal support element has a fixing plate extending in a horizontal plane which interacts with counter elements on the intermediate element.

7. The treatment facility of claim 6, wherein the counter elements include upper counter elements and lower counter elements, the upper counter elements are configured to transmit compressive forces acting to the fixing plate and the lower counter elements are configured to transmit tensile forces to the fixing plate.

8. The treatment facility of claim 7, wherein at least one of the upper counter elements or the lower counter elements are, adjustable in a vertical direction, on the intermediate element.

9. The treatment facility of claim 8, wherein distal support element reduces forces on the proximal support element during operation.

10. The treatment facility of claim 1, further comprising:
a drive for rotation of the intermediate element about the vertical axis, the drive being an Omega drive.

11. The treatment facility of claim 4, wherein the lower end point of the scissor arm guided in the guide of the floor-level plate is in an area of the distal support element in a state in which the distance between the lower end point of the scissor arm guided in the guide of the floor-level plate and the ring gantry is reduced.

12. The treatment facility of claim 5, wherein the distal support element has a fixing plate extending in a horizontal plane which interacts with counter elements on the intermediate element.

13. The treatment facility of claim 12, wherein
the counter elements include upper counter elements and lower counter elements, the upper counter elements are configured to transmit compressive forces acting to the fixing plate and the lower counter elements are configured to transmit tensile forces to the fixing plate.

14. The treatment facility of claim 13, wherein at least one of the upper counter elements or the lower counter elements are, adjustable in a vertical direction, on the intermediate element.

15. The treatment facility of claim 14, wherein distal support element reduces forces on the proximal support element during operation.
